(19) United States
(12) Patent Application Publication
(10) Pub. No.: US 2015/0071893 A1
(43) Pub. Date: Mar. 12, 2015

(54) THERAPEUTIC PREPARATION AND PROCESS FOR PREPARING SIAD THERAPEUTIC PREPARATION

(71) Applicants: Andrea BIGNOTTI, (US); Chiara TURELLI, (US)

(72) Inventors: Andrea Bignotti, Ospitaletto (IT); Chiara Turelli, Castegnato (IT)

(21) Appl. No.: 14/382,893

(22) PCT Filed: Mar. 5, 2013
(86) PCT No.: PCT/IB2013/051739

§ 371 Date: Sep. 4, 2014

(30) Foreign Application Priority Data
Mar. 6, 2012 (IT) MI2012A000338

(51) Int. Cl. A61K 35/14; A61K 47/44; A61K 9/00; A61K 33/00

(52) U.S. Cl. CPC A61K 35/19; A61K 33/00; A61K 47/44; A61K 9/0041
USPC 424/93.72

(57) ABSTRACT

Provided is a therapeutic preparation including a substance including ozone and a proteic substance. Also provided are methods of treating a disorder in a bovid, including administering to a subject in need thereof, a therapeutically effective amount of the therapeutic preparation.

The present invention relates to a therapeutic preparation and preparation process thereof, of the type as recited in the preamble of the independent claims.

Preferably, the invention relates to a therapeutic preparation for the treatment of bovidae, such as a bovine (Bos taurus) or sheep (Ovis aries) and, more specifically, for the treatment of ailments of the mammary gland, preferably mastitis, of a bovine.

As known, mastitis consists of an inflammation of the udder caused by microorganisms which, penetrating inside the mammary gland through the nipple provoke a response of the immune system inside said mammary gland and thus cause physical, chemical and bacteriological changes in the milk.

For example, the onset of mastitis translates into a reduction in milk production which may cease entirely.

Above all, gradually as the level of inflammation increases, the chemical composition of the milk increasingly resembles that of the blood on account of an alteration in the permeability of the membranes, which facilitates the filtration of haematic components from the blood circulation system to the udder, and a reduction in synthesis activity by the secretory tissue.

As a result, in some cases, the breeder, on account of the aforesaid alteration is forced to treat the animal with antibiotics for a period of time and discard the milk produced by the animal during such time.

A significant problem is lastly the fact that mastitis is diagnosed late and consequently, quantities of bad milk may be accidentally mixed with normal milk and be put on sale leading to problems of an enteric nature or other diseases in humans.

To resolve the aforesaid problems and therefore cure cases of mastitis, currently breeders use antibiotics.

The prior art described above has several significant drawbacks.

In fact the use of antibiotics or hormones causes residues of such substances in milk and thus creates heath problems in the consumer.

In particular, the presence of antibiotic residues in milk means that these can enter the human food chain increasing health risks to consumers, on account of the allergic, or in any case harmful effects which such substances may have.

In addition, the residues of said antibiotics transferred to humans from foods may contribute to the selection of resistant bacteria in the individual who has consumed the contaminated food. In fact, over recent years the diffusion of antibiotic-resistant phenomena has become widespread, with possible risks to public health.

Another problem is therefore the fact that the milk produced when the bovine is treated with antibiotic must be discarded leading to economic losses for the breeder.

A further problem is the fact that the residues of antibiotics are also found in urine or other excrements which, dispersed in the environment, are sources of pollution.

Another drawback, of no less importance is the fact that antibiotics have a high cost and are not particularly efficient.

In this situation the technical purpose of the present invention is to develop a therapeutic preparation for the treatment of bovidae and a preparation process thereof able to substantially overcome the inconveniences mentioned above. Within the sphere of said technical purpose one important aim of the invention is to obtain a therapeutic preparation for the treatment of bovidae which does not determine the presence of residues in the milk harmful to humans.

Another important aim of the invention is consequently to make a therapeutic preparation for the treatment of bovidae which does not require discarding of the milk produced during the period of treatment.

A further aim of the invention is to devise a therapeutic preparation for the treatment of bovidae which is particularly efficient and characterised by reduced environmental impact.

A no less important purpose is to obtain a preparation process of such therapeutic preparation which is easy and economical to perform.

The technical purpose and specific aims are achieved by a therapeutic preparation for the treatment of bovidae and a preparation process thereof as claimed in the appended claims.

Preferred embodiments are described in the dependent claims.

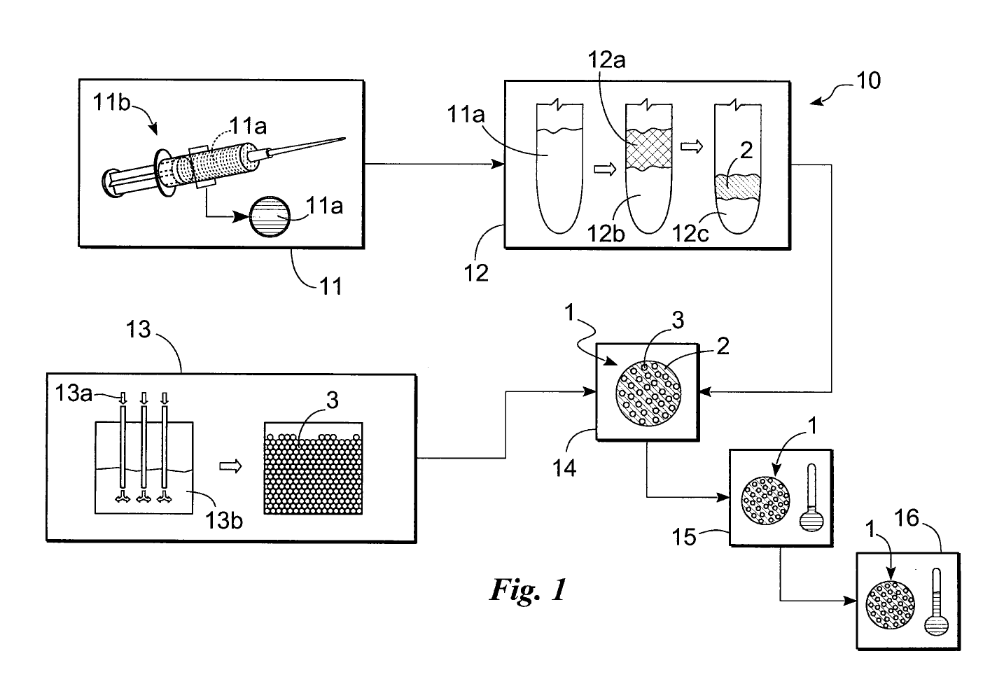

The characteristics and advantages of the invention are clearly evident from the following detailed description of a preferred embodiment thereof, with reference to the appended FIG. 1 showing a diagram of the preparation process of the therapeutic preparation for the treatment of bovidae according to the invention.

With reference to said drawings, reference numeral 1 globally denotes the therapeutic preparation according to the invention.

It is suitable to be used for the treatment and cure of external and internal inflammatory states both of a person and of an animal and, preferably of a bovine, that is an animal belonging to the Bovidae family. In particular, the therapeutic preparation 1 is suitable to be used in a bovine for the treatment of ailments of the mammary gland, articular inflammations with or without bacterial or infectious complications and for the regeneration of tissue to prevent tissue damage following injury, ageing. More in particular, the therapeutic preparation 1 is suitable to be used for the treatment of bovine (Bos taurus) mastitis.

The preparation 1 comprises, mainly, a substance of a proteic nature 2 and a substance including ozone 3 preferably in solution in a fluid. Said fluid is preferably a liquid and more preferably an oil, so as to make an ozonised oil 3.

The substance of a proteic nature 2 is a substance composed of amino acids and thus consists of monomers, that is, amino acids, dimers or proteic polymers.

Preferably the substance of a proteic nature 2 is a combination of growth factors. The term growth factors, in use in the medical field, is taken to mean a substance of a proteic nature suitable to stimulate and regulate the proliferation of cells.

By way of a non-exhaustive example, the main function of growth factors is the external control of the cellular cycle, through the abandonment of cellular quiescence (phase G0) and the entrance of the cell in phase G1 (of growth). Growth factors further regulate the activation of mitosis, cellular survival, migration and cellular differentiation. As well as proliferation they always contemporarily promote differentiation and maturation.

Again by way of a non-exhaustive example, a list of some growth factors is given below:
TGF-beta (transforming growth factor),
BMP (bone morphogenetic protein),
neurotrophins (NGF, BDNF and NT3),
FGF (fibroblast growth factor),
Macrophage Colony-Stimulating Factor (M-CSF) or CSF-1,
Granulocyte Macrophage Colony-Stimulating Factor (GM-CSF) or CSF-2,
Granulocyte Colony-Stimulating factor (G-CSF) or CSF-3,
nerve growth factor (NGF),
neurotrophins,
platelet derived growth factor (PDGF): located in the platelets and released by the α-granules thereof under various stimuli. It is also produced by macrophages. It also intervenes in the stabilisation of newly formed blood vessels, recruiting smooth muscle,
insulin-like growth factors,
erythropoietin (EPO),
thrombopoietin (TPO),
myostatin (GDF-8),
growth differentiation factor-9 (GDF9),
basic fibroblast growth factor (bFGF or FGF2),
epidermal growth factor (EGF) also known as epithelial growth factor, induces mitosis and can be found in various biological liquids (saliva, urine, sweat). It bonds to the EGFR receptor usually known as ERB-B1,
Hepatocyte growth factor (HGF),
vascular endothelial growth factor (VEGF): it is implicated in processes such as inflammation, angiogenesis, ischemic cells. There are different types such as: VEGF A, B, C, D, E which bond to receptors such as VEGFR1, 2, 3 which have different locations and bind different VEGFs. The VEGF induce an increase in the permeability of the blood capillaries, resulting in the formation of oedema,
TGF-α: Transforming growth factor-α implicated in almost all tumours. It bonds to the same receptor as the EGF and has the same effects,
TGF-β: Transforming growth factor-β, produced by platelets, macrophages, lymphocytes. It is synthesised in two forms, one latent and one active. The active form bonds first to the receptor2 forming a primary stable complex, which bonds to the receptor1, forming the secondary stable complex, which entails the phosphorylation of the SMAD transcription factors among which: SMAD2 and 3, which then bond to the SMAD4 transcription factor. A heterodimer results which is able to enter inside the nucleus and favour or inhibit gene activation. The TGF-β determines an increase in the concentration of factors inhibiting the CDK, causing blocking of the cellular cycle. It also intervenes in the stabilisation of newly formed blood vessels, recruiting matricellular proteins. Preferably the combination of growth factors is a platelet concentrate.

It is substantially composed of a haematic component, that is blood extract, the platelet concentration of which is preferably at least 10⁸ platelets/ml and appropriately, substantially equal to 10⁹ platelets/ml.

It is preferably of the allologous type, in other words obtained from a blood sample deriving from the same species, in particular bovidae or from the same breed including an animal which the preparation 1 is used on.

In another preferred solution the combination of growth factors is of the type contained in the Stem Cells Conditioned Medium.

The substance including ozone 3 is produced using any process, it may be pure ozone or ozone mixed with oxygen or not. Preferably it is ozone in solution in a fluid, or alternatively in a solid, and more preferably ozonised oil 3 which is substantially ozone emulsified with oil or similar substances and preferably emulsified with sunflower seed oil and, if required by specific applications, other additional components. It is composed of a gaseous liquid solution in which the ozone is preferably saturated.

The mixing ratio between the volumes of the substance of a proteic nature 2 and the substance including ozone 3, in the case in which the substance of a proteic nature 2 is a platelet concentrate and the substance including ozone 3 is ozonised oil is preferably substantially in the range between 2 and 4. Preferably, said mixing ratio is substantially in the range between 2.5 and 3.5 and, more preferably, close to 3, that is three parts of substance of a proteic nature 2 and one part of ozone.

The invention further relates to a process for preparing the therapeutic preparation described above for the treatment of bovidae.

Such preparation process 10 comprises a step 11 of taking a sample in which a sample of blood 11a is taken from the bovine; a separation step 12 in which the platelet concentrate 2 is extracted from the blood sample; an emulsifying step 13 in which the ozonised oil 3 is obtained by blowing ozone into the oil; a mixing step 14 in which the substance including ozone 3 and the platelet concentrate 2 are mixed; and a cooling step 15 in which the therapeutic preparation 1 is deep-frozen. In the step of taking a sample, the operator selects one or two bovidae and extracts a blood sample 11a. In particular, the blood sample 11a is extracted from healthy bovines, that is, characterised by a practically perfect state of health and thus free of the disease which the therapeutic preparation 1 is to address. In particular, during the step of taking a sample 11, the sample 11a is extracted for example with a syringe 11b through an outer jugular venipuncture or from the subcutaneous mammary vein, using a 16 gauge needle, substantially corresponding to 16.83 mm, and then collected in blood bags.

The blood sample 11a is then collected in bags made from PVC and containing an anti-coagulant and preservative additive such as, for example, CPDA-1. More in particular, the blood sample 11a is collected in blood bags which, for every 100 ml of blood substantially contain 0.327 g of monohydrate citric acid, 2.63 g of dihydrate sodium citrate, 0.251 g of dihydrate monosodium phosphate, 2.90 g of anhydrous dextrose and 0.0275 g of adenine.

Once the sample has been taken, step 11 is completed by placing the bags inside special refrigerators so as to keep them at a temperature substantially equal to 4° C.

Within twenty-four hours of taking the sample the process provides for a separation step 12 in which the substance of a proteic nature 2 is extracted from the blood sample 11a of the bovine. In detail, the blood is processed in such a manner as to obtain the substance of a proteic nature 2 characterised by a content of platelets substantially equal to 10⁹ platelets/ml.

In particular, in the step 12 the blood sample 11a is subjected to two centrifuging cycles, one at a low speed and one at a high speed, so as to avail of the different densities of the various elements present in the blood sample 11a. More in particular, the step 12 may involve a first centrifuging cycle at low revs (100 revs/min for 30 minutes) so as to separate the plasma rich in platelets 12b from the red blood cells or other discards 12c, a second centrifuging cycle at high revs (1500 revs/min for 10 minutes) in which the substance of a proteic nature 2 and the discard material composed of Platelet Poor Plasma (ppp) 12c is extracted from the plasma rich in platelets.

Lastly, should the substance of a proteic nature 2 thus obtained not have the desired platelet concentration, the separation step 12 provides for its dilution with Platelet Poor Plasma (ppp) 12c so as to obtain the desired concentration (10⁹ platelets/ml).

In the emulsifying step 13 the ozonised oil is prepared by blowing a mixture of oxygen-ozone 13a into oil 13b and preferably, into sunflower seed oil, preferably cold-pressed sunflower seed oil.

In detail, in such step 13 a sterile recipient 13c is partially filled with the oil 13b and then a mixture of oxygen-ozone at a concentration of 30 μg/ml is blown into it for 15 minutes so as to cause the emulsifying of the oil, and then the ozone 13b and the oil 13a are appropriately mixed to obtain the substance including ozone 3.

Once the emulsifying step 13 has been completed there is a mixing step 14 in which the substance including ozone 3 and the platelet concentrate 2 are mixed together.

In detail, in such step 14 the mixing ratio between the volumes of the content of the substance of a proteic nature 2 and of the content of the substance including ozone 3, preferably of the substance including ozone 3, is substantially in the range between 1 and 5. Preferably, said mixing ratio between the volumes of the concentrate 2 and of the ozonised oil 3 is substantially in the range between 2 and 4, more preferably in the range between 2.5 and 3.5 and, even more preferably, substantially equal to 3.

The cooling step 15 follows at this point in which the therapeutic preparation 1 is deep-frozen and in particular is brought to a temperature substantially equal to −80° C. for at least 12 hours or is treated with liquid nitrogen for a shorter period of time.

Such step is obtained for example by the mere collection of a sample using a syringe of a dose of platelet concentrate and a subsequent collection of a sample of ozone in the fluid. Given its inferior specific weight the ozone rises to the surface and remains partially in solution in the platelet concentrate which it has passed through.

In the cooling step 15, at the end of said 12 hours the therapeutic preparation 1 is thawed at a temperature in the range between +20° C. and 25° C. for 12 hours and then deep-frozen once again at −80° C. for 12 hours. Such step has the function of exploding the platelet cells which thus makes the necessary factors emerge. Alternatively, said step may be replaced or combined with the addition of calcium ions (Ca⁺ or Ca⁺⁺) which cause the said necessary factors to emerge from the platelet cells.

After completing the cooling step 15, the process involves a final preserving step 16, in which the therapeutic preparation is preserved until the moment of use at approximately −20° C.

The invention achieves some important advantages.

A first fundamental advantage is given by the innovative combination of a substance including ozone 3 and a substance of a proteic nature 2.

It achieves important advantages for the treatment of animals or people and as an antibiotic, anti-inflammatory product, antiseptic, tissue restructuring and regenerating product including the function of preventing tissue damage and/or the functional recovery of organ and/or tissues and/or following injury and/or disease and/or ageing.

In particular it is significantly advantageous as an antibiotic.

In detail it makes it possible to obtain an extremely efficient product for treating mastitis or other inflammations of the mammary gland.

Such aspect is also ensured by the cooling step 15 which, by bringing the preparation to −80° C. favours the release of the platelet factors characteristic of the preparation 1.

Moreover, the aforesaid combination makes the preparation 1 extraordinarily efficient in the treatment of a bovine or other animal or even humans as an antibiotic, anti-inflammatory product, antiseptic, tissue restructuring product for the treatment of external and internal inflammations, articular inflammation, bacteria and/or infectious complications, with a great tissue regenerating capacity and preventive function of tissue damage following injury or ageing.

One important advantage lies in the fact that, thanks to the particular composition of the therapeutic preparation 1, the milk produced during treatment with the therapeutic preparation 1 is free of residues of antibiotic and can thus be used by the consumer without any risk to health.

Another advantage is therefore the fact that, the use of the therapeutic preparation 1 does not require the milk producing during treatment to be discarded. Moreover, such advantage is increased by the fact that the therapeutic preparation 1, thanks to the combination of ozonised oil and platelet concentrate, does not involve a reduction in milk production.

A further advantage is the fact that, the concentrate 2 being of the allologous type, the preparation 1 is much better tolerated and practically free of side effects or adverse reactions.

Another advantage of no less importance is the reduced cost both of the therapeutic preparation 1 and of the process 10 compared to those currently known. A further important advantage is the extreme versatility of use of the therapeutic preparation 1 which, in fact, may be injected by means of a syringe, applied externally in the form of a cream (for example to the knee or a muscle) or internally (for example inside the uterus or mouth), or enclosed in a pill or the like to be swallowed by the bovine.

It is therefore easily utilisable for example in intramuscular, mammary intra-canalicular, intrauterine, transdermal, subcutaneous, intra-articular administrations but also by an enteral route, including oral, buccal or sublingual, rectal, therein including intrauterine and mammary intra-canalicular route, by a parenteral route among which intravenous, intramuscular, subcutaneous, inhalatory, intra-arterial, intrathecal, intraperitoneal, intra-articular and intradermal, locally or topically, among which cutaneous, transdermal, nasal, ophthalmic or auricular routes.

An experimental study of bovidae has verified the number of somatic cells present in a plurality of samples including milk from said bovidae taken before and after administration of the preparation 1 alone.

Said bovidae were treated with a daily dose, for four days in a row, of 6 ml of platelet concentrate 2 together with 2 ml of ozonised oil 3.

As known, the number of somatic cells present in milk is directly proportionate to the degree of infection of the related udder.

The result was as follows: on the day of administration the mean number of somatic cells present in the milk samples was approximately 9,658,182; seven days later they had decreased by 84% to a mean total of approximately 1,548,364 in the samples; a fortnight later the somatic cells had decreased by 97% to a mean total of approximately 276,455 in the samples; thirty days later the somatic cells had decreased by 99% to a mean total of approximately 125,364 in the samples. Another advantage is that the process 1 and the preparation 1 are practically free of pollutant agents.

1. A therapeutic preparation comprising: a substance comprising ozone and a proteic substance.

2. The therapeutic preparation as claimed in claim 1, wherein said proteic substance is a combination of one or more growth factors.

3. The therapeutic preparation as claimed in claim 2, wherein said proteic substance is a platelet concentrate.

4. The therapeutic preparation as claimed in claim 2, wherein said proteic substance is a medium conditioned by stem cells.

5. The therapeutic preparation as claimed in claim 1, wherein said substance comprising ozone is ozone in solution in a fluid.

6. The therapeutic preparation as claimed in claim 5, wherein said fluid is an oil.

7. The therapeutic preparation as claimed in claim 1, wherein said substance comprising ozone is ozonised oil.

8. The therapeutic preparation as claimed in claim 3,
wherein said substance comprising ozone is ozone in solution in an oil, and
wherein said platelet concentrate and said ozone in solution in an oil are present in said therapeutic preparation in a ratio of from about 2 to 4:1.

9. The therapeutic preparation as claimed in claim 1, for the treatment of bovine mastitis.

10. A process for preparing a therapeutic preparation for the treatment of bovidae, comprising:
mixing together a substance comprising ozone and a proteic substance, to produce a mixture.

11. The process according to claim 10, further comprising cooling the mixture to a temperature substantially equal to −80° C.

12. The process according to claim 10, further comprising freezing the mixture with liquid nitrogen.

13. The therapeutic preparation as claimed in claim 8, wherein said ozone in solution in an oil is ozonised oil.

14. The therapeutic preparation as claimed in claim 8, wherein said platelet concentrate and said ozone in solution in an oil are present in said therapeutic preparation in a ratio of from 2.5 to 3.5:1.

15. The therapeutic preparation as claimed in claim 8, wherein said platelet concentrate and said ozone in solution in an oil are present in said therapeutic preparation in a ratio of from about 3:1.

16. A method for treating a disorder in a bovid, comprising:
administering to a bovid in need thereof, a therapeutically effective amount of the therapeutic preparation as claimed in claim 1.

17. The method as claimed in claim 16, wherein said disorder is inflammation.

18. The method as claimed in claim 16, wherein said disorder is bovine mastitis.

19. The method as claimed in claim 19, wherein said ozone in solution in a fluid is ozonised oil.

20. The method as claimed in claim 16, wherein said proteic substance is a growth factor, a platelet concentrate, or a medium conditioned by stem cells.
